(19) United States
(12) Patent Application Publication
(10) Pub. No.: US 2022/0071573 A1
(43) Pub. Date: Mar. 10, 2022

(54) UPRIGHT ADVANCED IMAGING APPARATUS, SYSTEM AND METHOD FOR THE SAME

(71) Applicant: SOCIEDAD ESPANOLA DE ELECTROMEDICINA Y CALIDAD, S.A., Algete (ES)

(72) Inventors: José Maria Ortega, Madrid (ES); Juan Manuel Arco Casanova, Madrid (ES); Mónica Abella García, Madrid (ES); Manuel Desco Menéndez, Madrid (ES); Konstantin Sosenko, San Sebastian de los Reyes (ES)

(21) Appl. No.: 17/365,739

(22) Filed: Jul. 1, 2021

(30) Foreign Application Priority Data
Sep. 10, 2020 (ES) P202030920

(51) Int. Cl. A61B 6/02; A61B 6/00

(52) U.S. Cl. CPC A61B 6/025; A61B 6/4435; A61B 6/54; A61B 6/4476; A61B 6/4452

(57) ABSTRACT

Embodiments include a x-ray imaging system, apparatus, and method for use thereof. The apparatus comprises a first vertical column attached to a floor surface and configured to support an x-ray source; a second vertical column attached to the floor surface at a first distance opposite the first column and configured to support an x-ray imaging detector; and a positioning system configured to control vertical and angular movement of the x-ray source relative to the first column, wherein prior to image acquisition, the positioning system is configured to move the x-ray source to an initial height determined based on the detector height, and during image acquisition, the positioning system is configured to move the x-ray source to a plurality of positions along a trajectory defined by an upper angular position, a home position, and a lower angular position.

### CROSS-REFERENCE

This application claims the benefit of Spanish Patent Application No. P202030920, filed Sep. 10, 2020, the entire contents of which is incorporated by reference herein.

### TECHNICAL FIELD

This disclosure generally relates to imaging devices and more specifically, to techniques for acquiring images of the internal structures of a human body using advanced imaging technologies.

### BACKGROUND

Historically, x-ray diagnostic devices have been a commonly used modality to visualize the internal organs and structures of a patient in the field of healthcare. X-ray technology, itself, is based on the fundamental properties of the human body because each organ in a patient's body has its own physical characteristics, such as density and chemical composition, and the attenuation of an x-ray beam directed toward the patient depends on that density and chemical composition. For example, when the x-ray beam passes through the human body, organs with various density or chemical compositions absorb different amount of x-rays, and the resulting image obtained by the x-ray device represents distribution of that density or chemical composition inside the patient. This image is then used by a radiologist for diagnosis purposes.

Most conventional x-ray diagnostic systems utilize the basic principle of planar imaging using transmitted radiation to obtain x-ray images, as shown in FIG. 10. This type of system is easy to use, cost effective, and provides the projection image for diagnostic analysis in rapid time. At the same time, one disadvantage of this type of system is that the resulting image represents the “sum” of the images of all organs in the x-ray beam path, also known as “superposition.” In some clinical situations, this may affect the quality of the diagnostics, particularly in cases of lung abnormalities, such as nodules, pneumonias of various types, etc.

Further development of x-ray technology, especially the development of digital detectors of x-ray radiation (i.e. a device that converts the intensity of an incoming x-ray radiation into a digital signal), led to another type of x-ray system: computed tomography (CT) systems. Geometry of a typical CT system is shown in FIG. 11. The resulting image of a CT system is a three-dimensional image, which significantly improves the quality of diagnostics by reducing superposition of the internal organs. At the same time, this type of system has its own disadvantages, such as high cost, significantly higher amount of ionizing radiation dose to the patient, and relatively large dimensions, due to the need to turn the detector and x-ray tube assembly a full 360 degrees, or at least 180 degrees.

In 1931, linear tomography was introduced in clinical practice, making it one of the earliest imaging techniques to overcome the “superposition” issue of classical radiography. According to this technique, the x-ray tube is moved through a limited acquisition angle, with continuous emission of x-ray beams. On the resulting image, objects in the particular plane of interest (focus plane) are represented more clearly, while objects outside of the focus plane are blurred. This technique was mainly used for the analysis of pulmonary diseases, such as tuberculosis, calcifications in pulmonary nodes and lymph nodes, diseases of the sternum and central airways, etc. One limitation of this approach is the persistence of residual blur caused by objects in front of and behind the focus plane, often hiding soft tissue abnormalities, which leads to low contrast in the acquired image. Furthermore, to acquire the image of another focal plane, the whole procedure must be repeated, which means significant increase of radiation dose to the patient.

With the availability of digital flat panel detectors, the development of digital tomosynthesis (DTS) became possible. Generally, the DTS principle combines all of the above mentioned technologies. Namely, several classical projection images are obtained by positioning the x-ray tube at different angles (normally, a lot fewer than the number of angles required for CT), and the acquired images are processed so as to generate a set of planar images (or slices) representing a certain area (or section) of the patient anatomy, as shown in FIG. 12. In addition to eliminating the overlap of adjacent structures seen in classical x-ray images, thus effectively eliminating the superpositioning effect, DTS provides higher resolution in the coronal plane and a lower radiation dose than CT.

However, existing DTS systems are expensive and large, typically requiring an entire installation room due to its construction constrains. For example, the basic components of a DTS system are similar to those of a digital radiography system: an x-ray tube to emit ionizing radiation, a high voltage generator to supply electrical power to the x-ray tube, a flat panel digital x-ray detector, an anti-scatter grid, and mechanical components to properly hold and align the above mentioned components. In order to acquire images of the patient from several different angles, as is required for DTS image acquisition, a motorized crane is suspended from the ceiling and used to house and maneuver the x-ray tube to various positions, as shown in FIG. 13. In particular, the computer-controlled crane tilts the x-ray tube to preset angles as it follows a defined path relative to the detector, and the DTS system acquires images along the way. Commercially-available DTS systems, like the one shown in FIG. 13 serve their purpose, but the ceiling suspension aspect requires permanent installation in a dedicated radiology room, which increases the cost of acquisition and installation, limits the overall availability of the system, and prevents such DTS systems from being a significant alternative to existing CT systems.

In other diagnostic methods, such as dynamic imaging (e.g., fluoroscopy), x-rays play a different role. Specifically, in such mode, the x-ray detector captures multiple frames per second, which are then displayed to the radiologist as moving images, like an x-ray movie. Using available information about how internal organs typically move, it is possible to increase the sensitivity of the x-ray systems to several diseases. Initially, the resulting information could only be displayed in real time as the images are acquired with the patient next to the system. Further development of this technology has enabled the information acquired in dynamic imaging mode to be stored and reproduced at a later time, e.g., when required by a radiologist, without requiring the patient to the present.

As mentioned above, each organ in the patient's body has its own physical characteristics, such as density and chemical composition. The various types of x-ray systems described above use variation in density to generate diagnostically valuable information. It is also possible to acquire information related to the chemical composition of the organs by varying the spectra of an incident x-ray beam using a process known as spectral imaging. As an example, dual-energy imaging systems use only two different spectra to obtain diagnostic information, while multi-energy imaging systems use three or more incident x-ray spectra.

More specifically, it is known that the amount of x-rays that are absorbed by a given matter depends on the chemical composition of the matter, and this dependence has a non-linear character. Also, the absorption depends on the energy of the x-ray photons passing through the matter, which also has a non-linear character. Thus, by taking several images of the object with different x-ray energies, it is possible to measure the average atomic number of the object. This principle is used in conventional dual-energy x-ray diagnostic systems in order to, for example, mask organs or structures with specific atomic numbers. For example, bones, which contain a significant amount of calcium, can be masked to help diagnose soft tissues, or the reverse may be done, i.e. display just the bone structure to help diagnose bone fractures.

Existing dual-energy imaging systems come in various forms, and recent developments in detector technology have improved the speed and quality of dual-energy image acquisition. However, dual-energy imaging principles are typically applied to either existing CT installations, which are expensive and require a lot more space than conventional x-ray systems, or conventional x-ray systems, which are unable to avoid tissue superposition and thus, are limited in their sensitivity.

Thus, there are multiple technologies available for advanced imaging of the internal structures of the human body for diagnostic purposes, but each technology is realized in a very different type of system. Moreover, some of the described technologies are far from compact, which limits use of the systems to large dedicated rooms in hospitals and other large scale institutions.

Accordingly, there is still a need in the art for an improved x-ray-based diagnostic system that is capable of the imaging technology that is most appropriate for diagnosing a given case, but can still be compact and cost effective like a conventional x-ray system.

### SUMMARY

The invention is intended to solve the above-noted and other problems through systems, methods, and apparatus configured to (1) provide an upright, or floor-mounted, advanced imaging device comprising a first vertical column for supporting an x-ray imaging detector and a second vertical column for supporting an x-ray source (e.g., x-ray tube), the two columns being configured for placement in examination rooms where existing digital tomosynthesis (DTS) systems cannot be installed, including, for example, temporary spaces created for remote medical camps; (2) use digital tomosynthesis (DTS) to acquire images of the internal structures of a patient; and (3) be capable of using other imaging techniques, in addition to, or instead of, DTS, so as to allow selection of the best diagnostic modality for a given scenario.

For example, one embodiment provides an x-ray imaging apparatus comprising an x-ray source for emitting an x-ray beam towards a center of an x-ray imaging detector; the x-ray imaging detector configured to acquire an x-ray image of a patient positioned adjacent to the x-ray imaging detector and at least partially within a path of the x-ray beam; a first vertical column attached to a floor surface and configured to support the x-ray source; a second vertical column configured to support the x-ray imaging detector and attached to the floor surface at a first distance opposite the first vertical column, the x-ray image detector being adjustably positioned along an extent of the second vertical column at a detector height configured to substantially align with a target area of the patient; a positioning system configured to control vertical and angular movement of the x-ray source relative to the first vertical column, wherein prior to image acquisition, the positioning system is configured to move the x-ray source to an initial height determined based on the detector height, and during image acquisition, the positioning system is configured to move the x-ray source to a plurality of positions along a trajectory defined by an upper angular position, a home position, and a lower angular position.

Another exemplary embodiment provides an x-ray imaging system comprising an x-ray emission device comprising an x-ray source for emitting an x-ray beam towards a center of an x-ray imaging detector; an x-ray detection device comprising the x-ray imaging detector for acquiring an x-ray image of a patient positioned adjacent to the x-ray imaging detector and at least partially within a path of the x-ray beam; a positioning system configured to control vertical and angular movement of the x-ray emission device; a control unit configured to send control signals to the positioning system during image acquisition to move the x-ray emission device along a curvilinear trajectory about the x-ray imaging detector; and an x-ray generator configured to provide high voltage pulses of two or more different energy levels to the x-ray source for generating the x-ray beam, the x-ray generator being further configured to change from a first energy level to a second energy level while the positioning system moves the x-ray emission device from one position along the trajectory to a next position along the trajectory.

Yet another exemplary embodiment provides a method comprising: setting a detector height for an x-ray imaging detector supported by a detector column attached to a floor surface, the detector height configured to substantially align with a target area of a patient positioned adjacent the x-ray imaging detector; causing an x-ray source to move along a source column to an initial height, the initial height corresponding to the height of the x-ray imaging detector, wherein the source column supports the x-ray source and is coupled to the floor surface at a first distance opposite the detector column; causing the x-ray source to emit an x-ray beam towards a center of the x-ray imaging detector while the patient is positioned at least partially within a path of the x-ray beam; acquiring an x-ray image of the patient using the x-ray imaging detector; and during said acquiring, causing the x-ray source to move between a plurality of positions along a curvilinear trajectory defined by an upper angular position, a home position, and a lower angular position.

As will be appreciated, this disclosure is defined by the appended claims. The description summarizes aspects of the embodiments and should not be used to limit the claims. Other implementations are contemplated in accordance with the techniques described herein, as will be apparent to one having ordinary skill in the art upon examination of the following drawings and detail description, and such implementations are intended to within the scope of this application.

### BRIEF DESCRIPTION OF THE DRAWINGS

For a better understanding of the invention, reference may be made to embodiments shown in the drawings identified below. The components in the drawings are not necessarily to scale and related elements may be omitted, or in some instances proportions may have been exaggerated, so as to emphasize and clearly illustrate the novel features described herein. In addition, system components can be variously arranged, as known in the art. Further, in the drawings, like reference numerals designate corresponding parts throughout the several views.

FIG. 1 is schematic diagram of an exemplary upright advanced imaging apparatus, in accordance with certain embodiments.

FIG. 2 is a schematic diagram of the upright advanced imaging apparatus of FIG. 1 with a patient positioned between a detector and an x-ray source, in accordance with certain embodiments.

FIG. 3 is a schematic diagram of the upright advanced imaging apparatus of FIG. 1 implementing a first adjustment technique for accommodating a tall patient, in accordance with certain embodiments.

FIG. 4 is a schematic diagram of the upright advanced imaging apparatus of FIG. 1 implementing a second adjustment technique for accommodating a tall patient, in accordance with certain embodiments.

FIG. 5 is a block diagram of an exemplary upright advanced imaging system, in accordance with certain embodiments.

FIG. 6 is a schematic diagram of an exemplary beam filtration mechanism included in the upright advanced imaging system of FIG. 5, in accordance with certain embodiments.

FIG. 7 is a flow diagram of an exemplary method for carrying out a DTS mode of operation to obtain diagnostic images using the system shown in FIG. 5, in accordance with certain embodiments.

FIG. 8 is a flow diagram of an exemplary method for carrying out a multi-energy mode of operation to obtain diagnostic images using the system shown in FIG. 5, in accordance with certain embodiments.

FIG. 9 is a flow diagram of an exemplary method for carrying out a joint DTS and multi-energy mode of operation to obtain diagnostic images using the system shown in FIG. 5, in accordance with certain embodiments.

FIG. 10 is a schematic diagram of a conventional x-ray imaging system.

FIG. 11 is a schematic diagram of an existing computed tomography (CT) imaging system.

FIG. 12 is a schematic diagram of conventional digital tomosynthesis (DTS) being used to obtain multiple planar images.

FIG. 13 is a schematic diagram of an existing ceiling-mounted DTS system.

### DETAILED DESCRIPTION OF EXAMPLE EMBODIMENTS

While the invention may be embodied in various forms, there are shown in the drawings, and will hereinafter be described, some exemplary and non-limiting embodiments, with the understanding that the present disclosure is to be considered an exemplification of the invention and is not intended to limit the invention to the specific embodiments illustrated.

In this application, the use of the disjunctive is intended to include the conjunctive. The use of definite or indefinite articles is not intended to indicate cardinality. In particular, a reference to “the” object or “a” and “an” object is intended to denote also one of a possible plurality of such objects.

In the following description, elements, circuits and functions may be shown in block diagram form in order to not obscure the present disclosure in unnecessary detail. Additionally, block definitions and partitioning of logic between various blocks is exemplary of a specific embodiment. Further, those of ordinary skill in the art will understand that information and signals as depicted in the block diagrams may be represented using any variety of different technologies or techniques. For example, data, instructions, signals or commands may be represented in the figures, and which also would be understood as representing voltages, currents, electromagnetic waves or magnetic or optical fields, or combinations thereof. Additionally, some drawings may represent signals as a single signal for clarity of the description; and persons skilled in the art would recognize that the signal may represent a bus of signals. Various illustrative logic blocks, modules and circuits described in connection with embodiments disclosed herein may be implemented or performed with one or more processors. As would be appreciated and understood by persons of ordinary skill in the art, disclosure of separate processors in block diagrams may indicate a plurality of processors performing the functions or logic sequence disclosed herein, or may represent multiple functions or sequence performed on a single processor.

Systems, methods, and apparatus described herein provide upright advanced imaging technology that is more transportable and easier to install than existing advanced imaging systems, and has the capacity to increase diagnostic precision compared to classical or conventional x-ray systems. For example, an upright advanced imaging system described herein may be capable of using digital tomosynthesis (DTS) techniques to diagnose chest diseases, such as cancer or pneumonia, where conventional x-ray technology has shown limited sensitivity. Other advanced diagnostic capabilities may be provided by incorporating several x-ray-based diagnostic technologies into one system, such as, for example, DTS plus dynamic imaging and multi-energy imaging, and operating them either individually or jointly in order to acquire additional diagnostic information.

Moreover, unlike existing DTS systems, the upright imaging apparatus described herein uses a floor-mounted column to support the x-ray source (or tube), instead of a ceiling suspension system. This configuration provides a compact system design and the capacity for DTS image reconstruction from a scan of up to 50 degrees, for example. The floor-mounted or upright design also reduces the overall cost for equipment and makes the system, as a whole, easier to transport, install, use, and maintain. For example, the upright advanced imaging system described herein can be installed in a mobile unit (e.g., a truck or trailer), a relocatable enclosure (e.g., a shipping container), or other temporary examination room, thereby increasing healthcare availability to remote population groups, such as, e.g., in rural areas, refugee camps, military camps, etc.

In addition, the upright imaging apparatus described herein further comprises one or more automated position control systems configured to precisely and rapidly maneuver the x-ray source during image acquisition, as required to perform DTS. For example, each position control system may include one or more electronically controllable motors, drivers, and/or sensors to enable fine-tuned operation and synchronized movement.

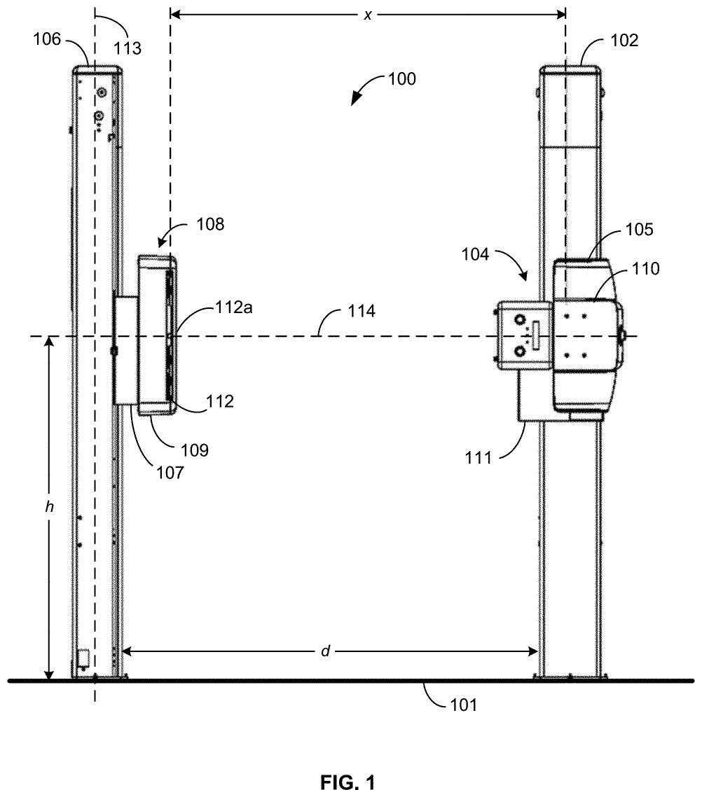

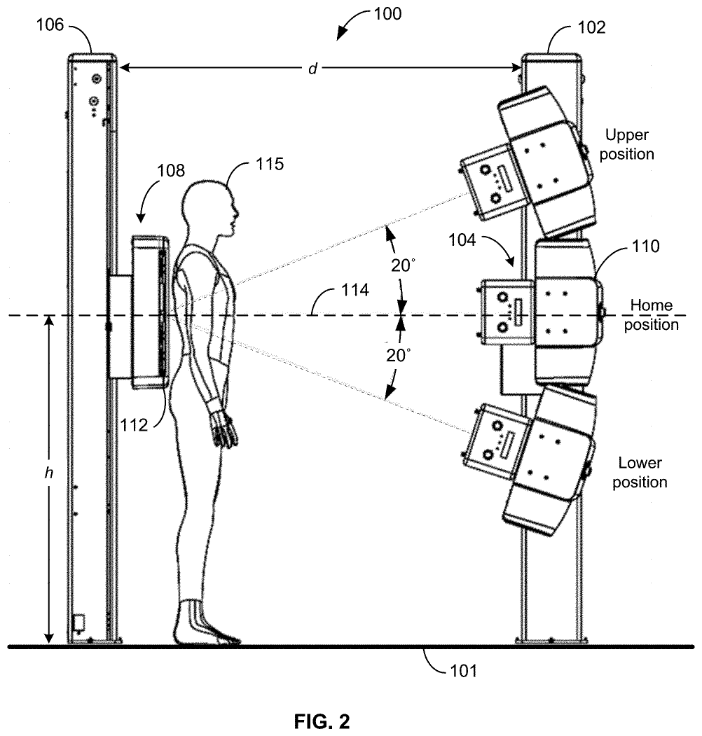

FIGS. 1 and 2 illustrate an exemplary upright advanced imaging apparatus 100 attached to a floor surface 101 and comprising a first column 102 for supporting and maneuvering an x-ray emission device 104 and a second column 106 for supporting and maneuvering an x-ray detection device 108, in accordance with embodiments. The x-ray emission device 104 comprises a first housing 105 and an x-ray source 110 (also referred to herein as an “x-ray tube”) for emitting ionizing radiation, or an x-ray beam, towards the center of an object to be imaged (e.g., a particular organ or region of the human body). In some embodiments, the x-ray emission device 104 further comprises an x-ray collimator (not shown) disposed adjacent the x-ray source 110 to limit the x-ray beam emitted towards the area of the patient body being imaged. In some embodiment, the x-ray emission deice 104 also comprises a dosimeter (not shown) for measuring the x-ray dose being provided to the patient. The x-ray detection device 108 comprises a second housing 109 and an x-ray imaging detector 112 (also referred to herein as a “detector”) positioned opposite, or facing, the x-ray tube 110 to obtain an x-ray image of the object placed in a pathway of the x-ray beam emitted from the x-ray source 110. In embodiments, the detector 112 may be a flat panel detector (FPD) or any other suitable x-ray imaging detector.

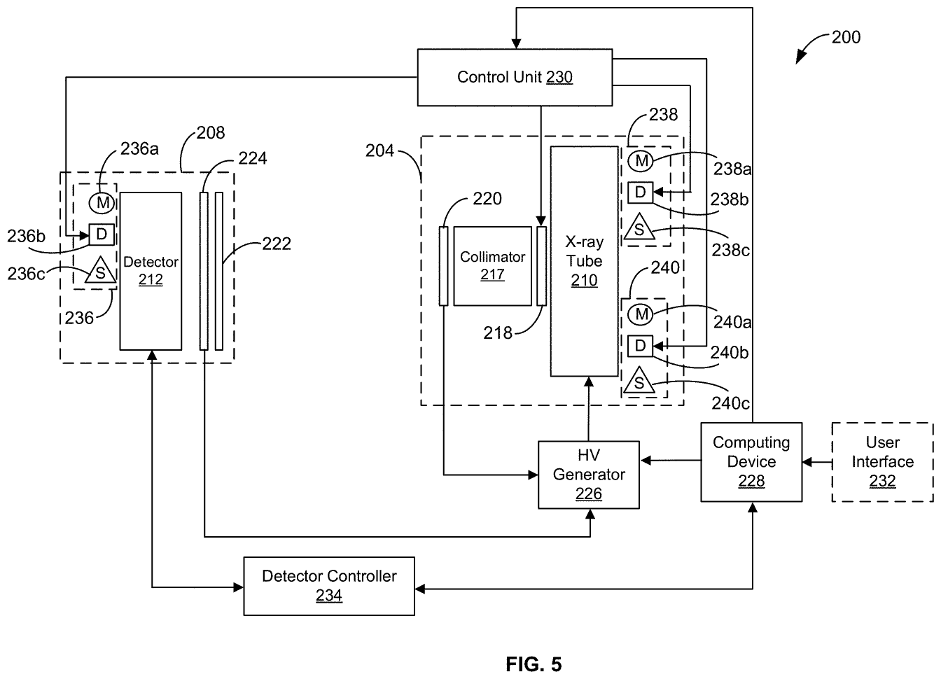

The first housing 105 (also referred to herein as a “source housing”) encases the x-ray source 110 and other components disposed within the device 104 (e.g., collimator and/or dosimeter) and can be configured to couple the x-ray emission device 104 to the first column 102 (also referred to herein as a “source column”). The source housing 105 may be coupled to, or include, a first positioning system 111 that is configured to rotatably and/or slidably connect the housing 105 to the source column 102 and control vertical and angular movement of the housing 105 relative to the source column 102. In embodiments, the first positioning system 111 comprises one or more computer-controlled devices (e.g., drivers, motors, and sensors shown in FIG. 5) configured to automatically move the x-ray source 110 along a curvilinear trajectory or path prescribed for image acquisition using DTS, or any other appropriate imaging technique, as well as move the x-ray source 110 to an initial height selected based on a height of the detector 112, as described herein. The term “vertical movement” is used herein to generally mean movement or travel along a vertical axis, or a motion directed up or down. The term “angular movement” is used herein to generally mean rotation about a fixed point or axis (e.g., a horizontal axis), or a motion directed to cause an angle between the object and the fixed axis to change.

The second housing 109 (also referred to herein as a “detector housing”) encases the x-ray detector 112 and other components disposed within the device 108 and can be configured to couple the x-ray detection device 108 to the second column 106 (also referred to herein as a “detector column”). The detector housing 109 may be arranged with, coupled to, or include, a second positioning system 107 that is configured to slidably connect the housing 109 to the detector column 106 and control vertical movement of the housing 109 relative to the detector column 106, or along a vertical axis 113 of the column 106. In embodiments, the second positioning system 107 can comprise one or more computer-controlled devices (e.g., drivers, motors, and sensors shown in FIG. 5) configured to automatically move the detector 112 to a desired height prior to imaging, for example, based on a height of a target area of the patient, as described herein.

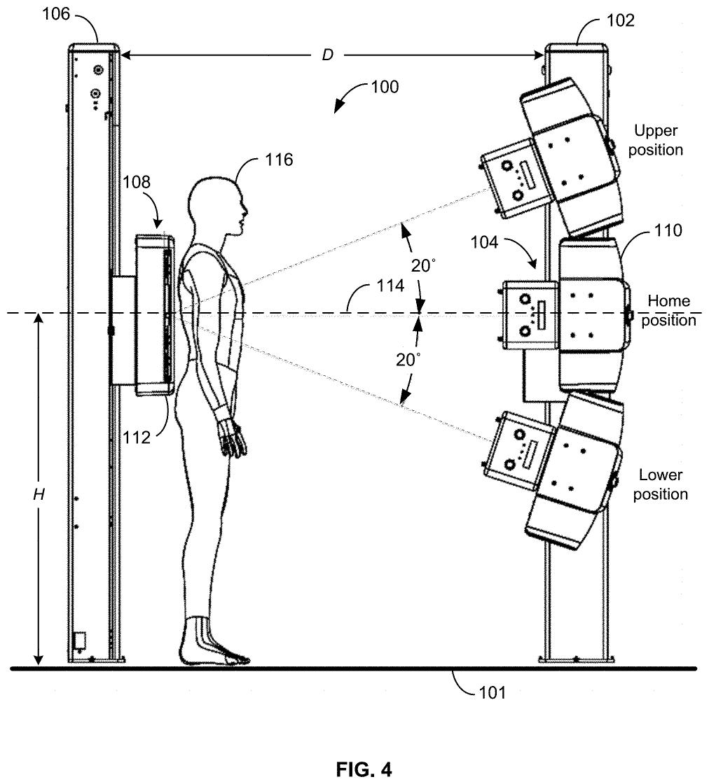

As shown, the source column 102 and the detector column 106 are positioned upright, or perpendicular to the floor surface 101, and spaced apart by a first distance, d. In addition, the columns 102 and 106 are positioned opposite each other and horizontally aligned, so that the detector 112 can be directed towards the x-ray source 110 as shown by central axis 114 in FIG. 1. In embodiments, the first distance d may be selected to ensure that the detector 112 is positioned a second distance, x, from the x-ray tube 110. The second distance x may be pre-defined based on the image acquisition mode selected for a given application, as will be appreciated. For example, the second distance x between the x-ray imaging detector 112 and the x-ray tube 112 may be prescribed based on an anatomy of the area to be imaged, a geometry of the x-ray beam being used, and/or the physics involved in the overall x-ray process. In some embodiments, the first distance d may be adjustable according to a height of the target area, or the area on the patient's body to be imaged (for example, as described below and shown in FIG. 4). Each of the columns 102 and 106 may be secured to the floor surface 101 (also referred to herein as a “floor”) using appropriate mechanical fasteners (e.g., bolts and screws). In some embodiments, the floor surface 101 may be a base or lower surface of the advanced imaging apparatus 100. In other embodiments, the floor surface 101 may be the floor of an examination room located in a healthcare institution (e.g., a hospital or clinic) or the floor of a makeshift or compact examination room located in a mobile or relocatable medical facility. For example, the floor surface 101 may be the floor of a medical truck or trailer, or the floor of a shipping container configured for examination use. Accordingly, the columns 102 and 106 may also be configured for removable attachment to the floor surface 101, to enable the apparatus to be moved to, and installed in, another location, as needed.

Each of the first column 102 and the second column 106 can be configured or constructed to support a weight of the x-ray emission device 104 and the x-ray detection device 108, respectively, as well as that of cables, pulleys, trolleys, and/or any other mechanisms or devices coupled to each column 102, 106 to enable movement of the devices 104 and 108 along their respective columns. For example, the columns 102 and 106 may be made of a sturdy material, such as metal, and have appropriate dimensions (e.g., height, width, length, thickness, etc.) and an appropriately weighted base selected to maintain the columns 102 and 106 in an upright position while supporting the devices 104 and 108, respectively.

An overall height of the source column 102 may be selected to accommodate a path or distance travelled by the x-ray emission device 104 as it moves between the various angles required for DTS image acquisition, or other image acquisition protocol. Likewise, an overall height of the detector column 106 may be selected to accommodate a vertical displacement of the x-ray detection device 108 when adjusting a height, h, of the detector 112, for example, to substantially align with a height of the target area of the patient. Though the illustrated embodiment shows the two columns 102 and 106 as being of equal, or substantially equal, height, in other embodiments, the detector column 106 may be shorter in height than the source column 102 because, for example, due to the source angles required during DTS image acquisition, the requisite height, h, of the detector 112 for any given patient may always be lower than the upper most position of the x-ray source 110.

During operation of the apparatus 100, a patient 115 is positioned adjacent to a front surface 112a of the detector 112 as x-ray beams are directed from the x-ray tube 110 towards the patient 115, as shown in FIG. 2. At an initial stage, or before image acquisition begins, a height of the x-ray detection device 108 is adjusted based on the height of a target area of the patient 115. In particular, the x-ray detection device 108 may be moved vertically along the second column 106 until the detector 112 is at a desired height, h, that substantially aligns with the target area of the patient 115, or the body area to be imaged. For example, in FIG. 2, the detector 112 is aligned with a center of the patient's chest in order to obtain images of the patient's chest area. In some embodiments, the height of the detection device 108 is manually adjusted or set by an operator of the apparatus 100. In other embodiments, the height of the detection device 108 is automatically set by the apparatus 100. Once the detection device 108 is moved to a desired height, a height of the x-ray emission device 104 is automatically adjusted by the apparatus 100 in accordance with the height of the detection device 108 and a selected imaging protocol. For example, the x-ray emission device 104 may be moved vertically along the first column 102 until a center of the x-ray tube 110 is aligned with a center of the detector 112, or the central axis 114, as shown by “home position” in FIG. 2.

During image acquisition, the x-ray emission device 104 may be moved both angularly and vertically relative to the central axis 114 and the source column 102 to enable the x-ray tube 110 to move along a path prescribed by the selected image acquisition mode. The x-ray emission device 104 may pause at predefined locations along the prescribed path in order to emit the x-ray beam towards the detector 112 from various angles. Each image is obtained while the x-ray beam is directed at the center of the detector 112. Thus, the number of angles may be selected based on the number of images, or slices, desired for a given application.

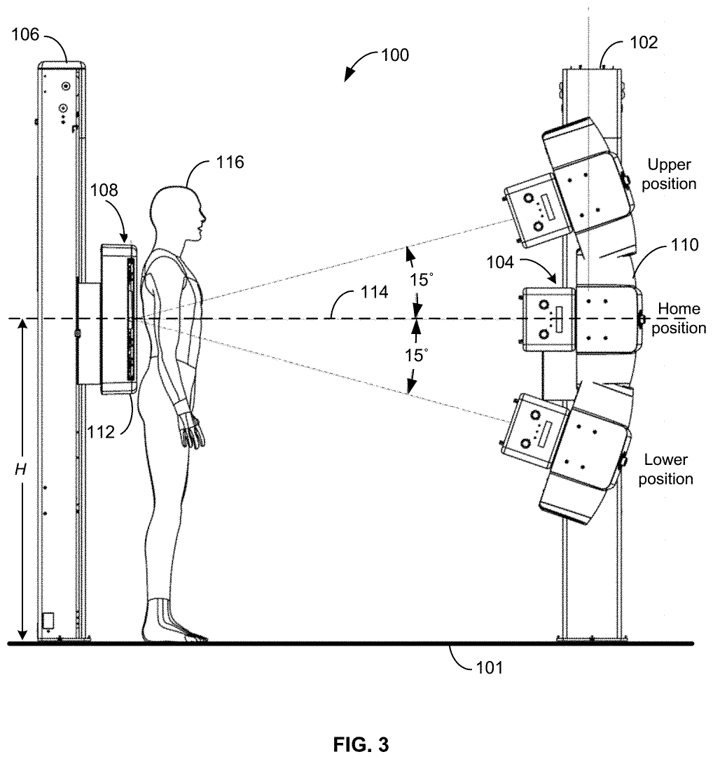

As an example, FIG. 2 shows three possible positions for the x-ray emission device 104 during DTS image acquisition: a home or rest position for emitting the x-ray beam towards the center of the detector 112 from a 0 degree angle (or along the central axis 114), such that the beam is perpendicular to the front surface 112a of the x-ray detector 112; an upper angular position for emitting the x-ray beam towards the center of the detector 112 from a +20 degree angle relative to the central axis 114; and a lower angular position for emitting the x-ray beam towards the center of the detector 112 from −20 degree angle relative to said central axis 114. The three positions may define the path traveled by the x-ray emission device 104 during image acquisition, while the upper and lower positions may limit a total angular range of movement of the x-ray tube 110. In embodiments, the home position may be determined first, depending on the height, h, selected for the detector 112 based on a patient height and/or a location of the area to be imaged. Then the upper and lower angular positions may be determined by mapping out the upper and lower DTS acquisition angles relative to, or starting from, the home position. As shown, the upper and lower angles may be equal in magnitude but extend in opposite directions. The angle values, or the total angular range of movement, may be selected based on a desired resolution for the resulting projection images and/or a distance, x, between the detector 112 and the x-ray tube 110. In the illustrated embodiment, the total angular range of movement is limited to about 40 degrees due to a DTS acquisition angle of +/−20 degrees. In other embodiments, the total angular movement may be greater or less than 40 degrees, depending on the exact DTS acquisition angle selected (e.g., as shown in FIG. 3).

In embodiments, the overall height of the source column 102 may be selected based on an upper-most positioning of the x-ray emission device 104 during image acquisition. For example, in FIG. 2, the depicted upper angular position may determine a minimum height requirement for the source column 102. However, as described above, the upper angular position is dependent on a height of the target area of the patient, since the detector height h is adjusted based on the target height. Thus, for example, the taller a patient is, the higher up the upper angular position will be. As will be appreciated, if the upright imaging apparatus 100 is designed to accommodate patients of all heights, including those that are very tall (e.g., above two meters), an overall height of the apparatus may exceed a ceiling height of certain examination rooms (e.g., medical trucks, vans, trailers, or containers), or may otherwise negate the compact-size advantages of the upright imaging apparatus 100 described herein. For this reason, in various embodiments, the upright imaging apparatus 100 may be configured to use one or more adjustment techniques for accommodating patients of different heights.

According to a first adjustment technique (or algorithm), the upright imaging apparatus 100 is configured to vary a DTS acquisition angle of the x-ray tube 110 based on the height of a given patient, and an overall height of the source column 102 can be selected based on the upper angular position required to accommodate the tallest patient supported by the apparatus 100. For example, in some embodiments, the DTS acquisition angle may be selected from a range of about 12 degrees to about 25 degrees, depending on the patient height and/or a height of the location to be imaged (i.e. the target area), and the total angular range of movement for the x-ray tube 110 may vary between about 24 degrees and about 50 degrees, depending on the selected angle. In such embodiments, the overall height of the source column 102 may be selected upon determining the upper angular position required to implement a DTS acquisition angle of 12 degrees, or the angle designed to accommodate an upper limit to patient height.

FIG. 3 illustrates an exemplary implementation of the first adjustment technique using the apparatus 100. In particular, the upright imaging apparatus 100 has been configured for use by a second patient 116 that is taller than the first patient 115 shown in FIG. 2 (e.g., taller than 2 meters), without increasing the overall height of the source column 102. As shown, the detector 112 has been moved upwards to a second selected height H that is based on the second patient height and is greater than the initially selected detector height h required for the first patient 115 in FIG. 2. To accommodate the new detector height H, the apparatus 100 has reduced the DTS acquisition angle to about 15 degrees, from the 20-degree angle shown in FIG. 2. As a result, the upright imaging apparatus 100 is able to acquire appropriate DTS images of the second (taller) patient 116 while keeping the overall height of the source column 102 small or compact enough for mobile or relocatable applications, for example.

In other embodiments, the upright imaging apparatus 100 may be configured to utilize a second adjustment technique to accommodate tall patients without increasing the overall height of the source column 102. According to this technique (or algorithm), the apparatus 100 changes the distance, d, between the detector column 106 and the source column 102 based on the height of the target area of a patient, or the corresponding detector height, H, required for said patient, so that the DTS acquisition angle can remain constant (e.g., at about 20 degrees) for patients of all heights. For example, the distance between columns 102 and 106 may be reduced from the original distance, d, shown in FIG. 2, (e.g., about 180 centimeter (cm)) to a new distance, D, as shown in FIG. 4, (e.g., about 140 cm or 150 cm) to accommodate taller patients. In some cases, the column separation distance, d, may be increased to accommodate shorter patients.

In various embodiments, one or more of the columns 102 and 106 may be configured to move or slide horizontally in order to reduce, or otherwise change, the distance, d, between the columns. For example, the upright imaging apparatus 100 may include a track system, a sliding apparatus, one or more rails, and/or other suitable mechanism coupled to the floor surface 101 and one or more of the columns 102 and 106 for carrying out said movement. For example, the sliding mechanism may be included in, attached to, or placed on the floor surface 101. The upright imaging apparatus 100 may further include a third positioning system comprising computer-controlled devices (e.g., drivers, motors, and sensors, as described herein) for controlling said mechanism and enabling said movement in an automated manner. In some embodiments, the detector column 106 may be configured to move forward or towards the source column 102 and move back to an initial position along the same path, as needed. In other embodiments, the reverse may be true, additionally or alternatively; i.e. the source column 102 may be configured to move forward or towards the detector column 106 and move back to an initial position along the same path, as needed.

The apparatus 100 may further include a controller (not shown) configured to control movement of the column(s) based on inputs describing the patient height and/or the region of the patient to be imaged. For example, the controller may include software configured to determine or calculate the column separation distance, d, required for a given detector height, h, patient height, or target area height, and may be communicatively coupled to the third positioning system (not shown) and/or other mechanisms for moving the one or more columns as needed. In other embodiments, the controller may be configured to select between an original or preferred column separation distance (e.g., about 180 centimeters) and a reduced column separation distance (e.g., about 150 centimeters) depending on whether the patient 116 meets or exceeds a threshold height requirement (e.g., 2 meters), respectively.

In other embodiments, instead of moving the columns 102 and 106, at least one of the x-ray emission device 104 and the x-ray detection device 108 can be configured to move horizontally, relative to the given column 102/106, in order to increase and/or decrease a distance between the x-ray detector 112 and the x-ray tube 110. In such cases, the relevant device 104/108 may include one or more mechanisms for adjusting a horizontal distance between the device 104/108 and the corresponding column 102/106 or otherwise enabling said movement (e.g., a track, an extendable arm, a sliding apparatus, etc.). A controller and third positioning system, similar to those described above, may also be included to control said movement. FIG. 4 illustrates an exemplary implementation of the second adjustment technique using the upright imaging apparatus 100. As shown, the detector 112 has been moved up to a detector height, H, to accommodate a tall patient 116 (e.g., over 2 meters). In order to enable the x-ray emission device 104 to move along the path prescribed for DTS image acquisition while still aligning the x-ray source 110 with the higher detector height, H, the apparatus 100 has moved the columns 102 and 106 closer together to a column separation distance, D, that is less than the original distance d shown in FIG. 2. In some embodiments, the exact distance, D, may be selected so that the DTS acquisition angle of the x-ray tube 110 can remain fixed at 20 degrees. In other embodiments, the original column separation distance, d, may be preset at about 180 centimeters, and the reduced column separation distance, D, may be preset at about 150 centimeters for patient heights over 2 meters, for example. In this manner, the overall height of the source column 102 can remain as is, i.e. compact enough for mobile or relocatable applications, for example.

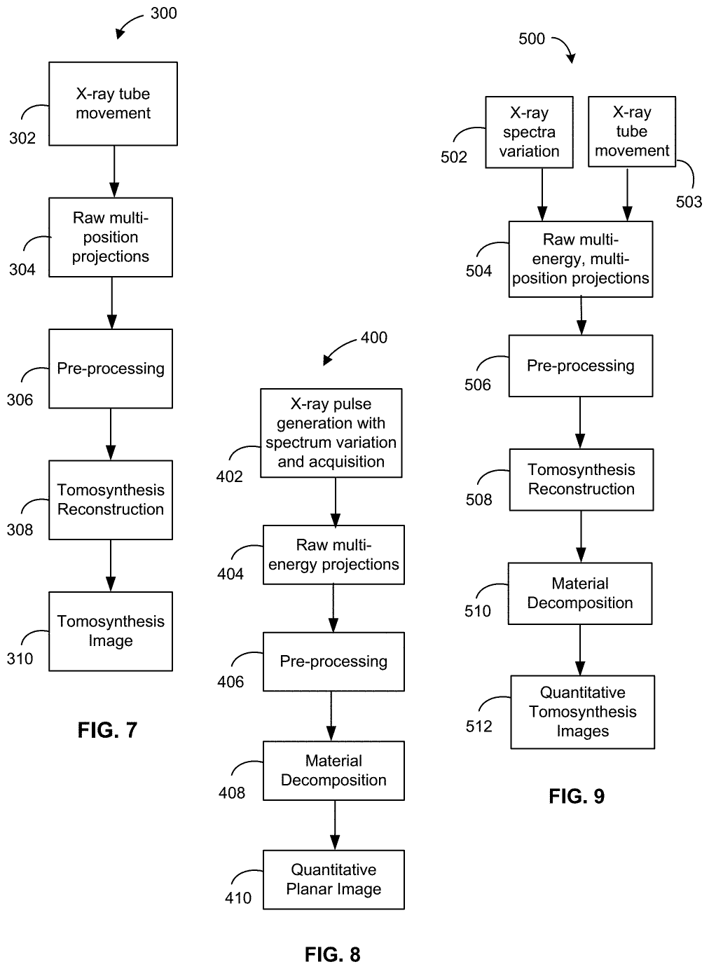

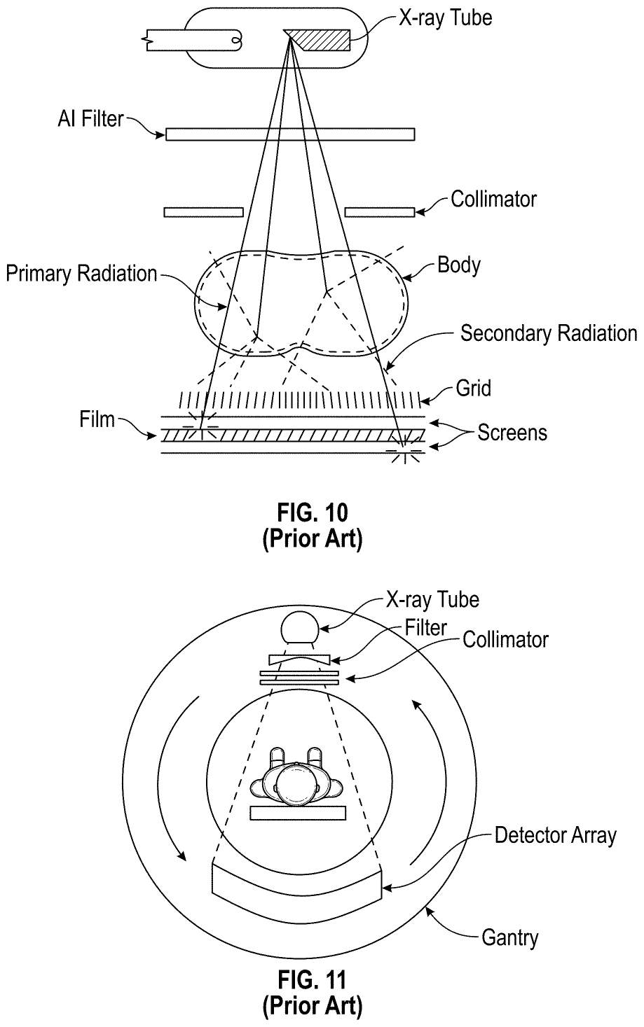

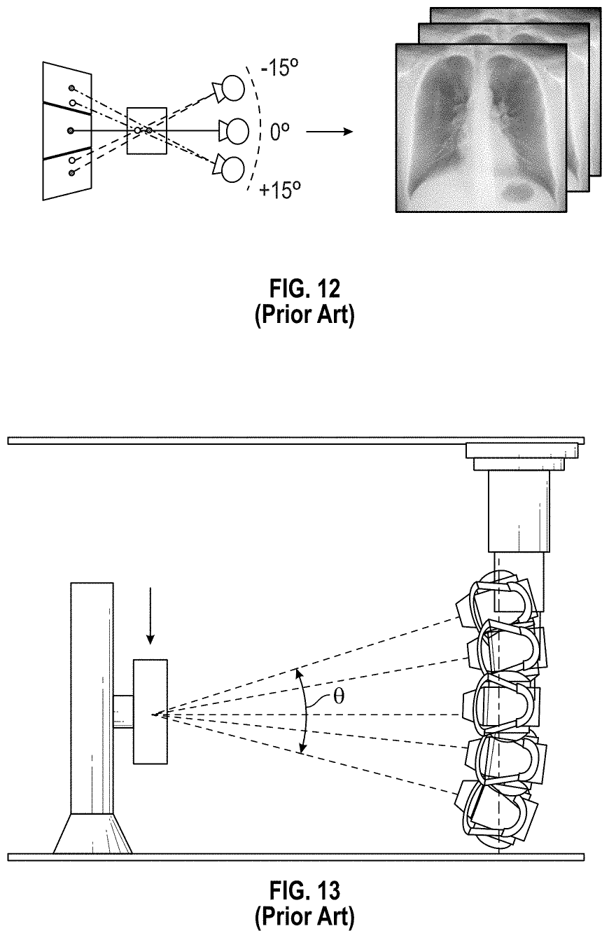

FIG. 5 is a functional block diagram of an exemplary upright advanced imaging system 200 (also referred to herein as “advanced imaging system”), in accordance with embodiments. The advanced imaging system 200 comprises an upright advanced imaging apparatus that is substantially similar to the upright imaging apparatus 100 shown in FIG. 1. For example, the advanced imaging system 200 comprises an x-ray emission device 204 that includes an x-ray tube 210, similar to the x-ray emission device 104 and tube 110 shown in FIG. 1. Likewise, the advanced imaging system 200 also comprises an x-ray detection device 208 that includes an x-ray imaging detector 212, similar to the x-ray detection device 108 and detector 112 shown in FIG. 1. Though not shown, the advanced imaging system 200 may also comprise a first vertical column (or source column) for supporting the x-ray emission device 204, similar to the source column 102 of FIG. 1, and a second vertical column (or detector column) for supporting the x-ray detection device 208, similar to the detector column 106 of FIG. 1. In embodiments, the advanced imaging system 200 can be configured to carry out one or more techniques for controlling and operating the upright imaging apparatus, such as, e.g., method 300 of FIG. 7, method 400 of FIG. 8, and/or method 500 of FIG. 9.

As shown, the x-ray emission device 204 further comprises a collimator 217 disposed adjacent to an output end (or emitting portion) of the x-ray tube 210. The collimator 217 can be configured to minimize the field of radiation to avoid unnecessary irradiation of a patient's body. In particular, the collimator 217 limits or narrows a size of the x-ray beam being directed towards the patient as it exits the x-ray source 210. The specific size of the x-ray beam may be determined based on the target area, or the area to be imaged on the patient's body (e.g., a particular organ or region of the body). As an example, the collimator 217 may comprise a series of metal leaves or blades (e.g., tungsten) that overlap to create different-sized openings or fields. In embodiments, an opening of the collimator 217 can be automatically, or manually, adjusted according to a size of the detector 212, such that the portion of the x-ray beam that reaches the detector 212 generally coincides in size with that of the overall detector 212.

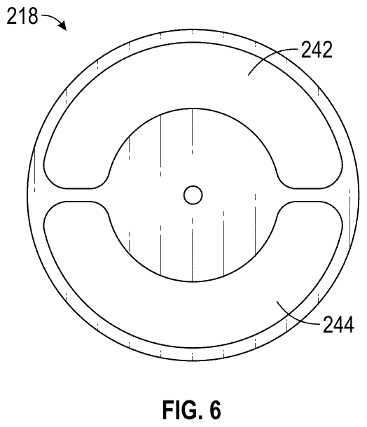

In some embodiments, the x-ray emission device 204 further comprises a beam filtration mechanism 218 positioned between the collimator 217 and the output end of the x-ray source 210. The beam filtration mechanism 218 can be configured to position filtration material over or before the x-ray beam being emitted by the x-ray tube 110 in order to change an energy level of the beam, as described in more detail with respect to FIG. 6 below.

As shown, the x-ray emission device 204 can also comprise a dose-area-product (DAP) meter 220 disposed adjacent an output end of the collimator 217 to measure an amount of ionizing radiation that falls on or reaches the patient. In some cases, the x-ray emission device 204 also includes one or more filters (not shown) for removing any unnecessary or unusable parts of the x-ray output produced by the x-ray source 210.

As shown, the detector 212 may be a flat panel detector (FPD) or any other suitable x-ray imaging detector. The x-ray detection device 208 further comprises an anti-scatter grid 222 positioned between the detector 212 and an object being imaged (e.g., the patient) in order to remove secondary (or scattered) radiation from the incident beam, thus ensuring that only the primary beam, or the portion of the beam that contains useful information, reaches the detector 212. The x-ray detection device 208 can also comprise an automatic exposure control (AEC) chamber 224 configured to help maintain the dose of ionizing radiation at a desired level.

The upright imaging system 200 further comprises an x-ray generator 226 (also referred to herein as a “high voltage generator” or “HV generator”) for providing high voltage power, or pulses, to the x-ray tube 210 for generating the x-ray beam. As shown in FIG. 5, the HV generator 226 may be electrically connected to the AEC chamber 224 and the DAP meter 220 as well. In embodiments, the AEC chamber 224 and/or the DAP meter 220 may send a signal to the HV generator 226 to stop delivery of the high voltage power (or pulse) to the x-ray tube 210 once a necessary dose of radiation is reached.

During operation, the x-ray tube 210 generates an x-ray beam, or x-radiation, by converting electron energy into photons. More specifically, the x-ray tube 210 includes a cathode and an anode. As electrical current flows through the tube 210 from the cathode to the anode, the high tension between these two components causes electrons to accelerate, or travel at a high velocity, towards the anode. During this acceleration, the electrons receive or increase their energy. Upon striking the anode, the electrons undergo an energy loss, which results in the generation of x-radiation. The quantity (or exposure) and quality (or spectrum) of the resulting x-radiation can be controlled by adjusting certain parameters that control the x-ray production process (also referred to herein as “exposure control parameters”). These include the voltage or electrical potential (measured in kilo-Volts (kV)) that is applied to the x-ray tube 210 by the HV generator 226, the electrical current (measured in milli-Amps (mA)) that flows through the x-ray tube 210, and the exposure time or duration (measured in milli-seconds (mS)) of the x-ray tube 210. The electrical potential (kV) determines the amount of energy carried by each electron emitted from the cathode, and the electrical current (also referred to herein as “anode current”) determines the number or quantity of electrons that strike the anode.

The x-ray beam generated by the x-ray tube 210 first passes through the beam filtration mechanism 218, then through the collimator 217, and finally through the DAP meter 220, before exiting the x-ray emission device 204. Once outside the device 204, the x-ray beam goes through the patient (e.g., patient 115 in FIG. 2), and is attenuated along the way by the internal structures or organs of the patient. After exiting the patient, the x-ray beam enters the x-ray detection device 208, first passing through the anti-scatter grid 222 and then through the AEC chamber 224, before finally reaching the detector 212. The detector 212 converts the x-ray beam into an electrical signal, wherein the value of the signal is proportional to an intensity of the x-ray beam.

According to embodiments, the advanced imaging system 200 further comprises one or more controllers, control modules, and other components comprising circuitry or electronics configured to control specific aspects of the above image acquisition process, or more specifically, parameters of the x-ray emission device 204 and the x-ray detection device 208. In particular, the advanced imaging system 200 includes a computing device 228 (e.g., computer) configured to control various aspects of the system 200, a control unit 230 (e.g., controller) communicatively coupled to the computing device 228, and a user interface 232 communicatively coupled to the computing device 228 for enabling user control of various settings of the system 200. The control unit 230 can be configured to govern the overall operation of the upright imaging apparatus, for example, based on instructions received from the computing device 228 and/or commands received from the user via the user interface 232 (e.g., start exposure, stop exposure, etc.). In embodiments, the control unit 230 may include a processor and memory configured to carry out these instructions and/or commands. The computing device 228 can be configured to set or adjust the parameters of the control unit 230 that are used to control operation of the upright imaging apparatus, including synchronizing movement of the x-ray tube 210 and the detector 212, for example. In some embodiments, the computing device 228 may also receive or acquire demographic information associated with the patient from a hospital network or other database.

In addition, the advanced imaging system 200 comprises a detector controller 234 communicatively coupled to the detector 212 as well as the computing device 228, as shown. The detector controller 234 can be configured to control operation of the detector 212, process signals received from the detector 212, and provide resulting information, including x-ray images, to the computing device 228. As an example, the detector controller 234 may receive a signal from each element of the detector 212 that is exposed to the x-ray beam and acquire an image based thereon, in accordance with instructions received from the computing device 228. The computing device 228 can be configured to process the information received from the detector controller 234, including any image information. In some embodiments, the computing device 228 may include an image processor for processing the x-ray imaging signal provided by the detector 212.

In embodiments, the computing device 228 can be configured to set or adjust parameters of the HV generator 226, such as, for example, the exposure control parameters for the high voltage pulses provided to the x-ray tube 210, based on control inputs received from the user interface 232, as well as other information. The HV generator 226 may include, or be coupled to, an exposure controller (not shown) for controlling operation of the HV generator 226 and the x-ray source 210 based on the received information. In particular, the exposure controller may be configured to generate an appropriate amount of x-ray exposure dosage based on instructions received from the computing device 228, such as, e.g., when to start or stop an exposure, what values to apply for the exposure control parameters of the x-ray source 210 (e.g., kV, mA, and mS), etc.

In embodiments, one or more of the HV generator 226, the computing device 228, the control unit 230, the user interface 232, and the detector controller 234 may be housed in one or more units that are separate from the x-ray emission device 204 and the x-ray detection device 208. For example, such unit(s) may be included on, or coupled to, one or more of the vertical columns of the upright imaging apparatus, or may be a standalone unit disposed near the vertical columns but external to the upright imaging apparatus. In either case, one or more cables, wires, or other suitable connection mechanisms, including wireless connections (e.g., WiFi, Bluetooth, RFID, etc.), may be used to communicatively couple the components of the system 200 to each other, as needed, for example, to ensure that instructions from the computing device 228 are appropriately received at the detector controller 234, HV generator 226, and control unit 230.

In some embodiments, the HV generator 226 may be disposed within the x-ray emission device 204, the detector controller 234 may be disposed within the x-ray detection device 208, and the computing device 228 may be disposed in a standalone unit that is communicatively coupled to the devices 204 and 208. In such embodiments, the user interface 232 may be disposed in the same standalone unit, and the control unit 230 may be disposed in either said standalone unit or in the x-ray emission device 204. In the latter case, the x-ray emission device 204 may be communicatively coupled to the x-ray detection device 208 (e.g., via wired or wireless connection) in order to transmit control signals from the control unit 230 to the x-ray detection device 208.

The user interface 232 can be configured to allow user control of various settings of the system 200, such as, e.g., x-ray tube current (mA) and voltage (kV) parameters, as well as exposure time (mS). In embodiments, the user interface 232 can include one or more input devices (e.g., a keyboard, a mouse, a touch screen, a microphone, a stylus, a radio-frequency device reader, one or more buttons, sliders, knobs, switches, and/or other tactile input devices, and the like) for receiving said user inputs. In some embodiments, the user interface 232 is integrated into the computing device 228. In other embodiments, the user interface 232 is a standalone device, such as, for example, an operating console, for enabling users to control the various settings of the system 200. In such cases, the user interface 232 may be communicatively coupled to the computing device 228 via a wired or wireless connection for providing the received inputs thereto. In some embodiments, the user interface 232 may include a display device (not shown) for displaying content to the user, such as, e.g., x-ray images obtained by the detector 212.

Though not shown, the computing device 228 comprises at least one processor and memory for implementing the techniques described herein. During operation of the computing device 228, the at least one processor can be configured to execute software stored within the memory, communicate data to and from the memory, and generally control operations of the computing device 228 pursuant to the software. In some embodiments, the computing device 228 further includes a communications module comprising one or more transceivers and/or other devices for communicating with one or more networks (e.g., a wide area network (including the Internet), a local area network, a GPS network, a cellular network, a Bluetooth network, other personal area network, and the like).

As an example, in some embodiments, the computing device 228 can be configured to, via the at least one processor executing software stored in the memory, perform a method for operating the advanced imaging system 200, the method comprising a plurality of steps, including setting a detector height for the x-ray imaging detector 212, wherein the detector 212 is supported by a detector column attached to a floor surface (e.g., as shown in FIG. 1), and the detector height is configured to substantially align with a target area of a patient positioned adjacent the x-ray imaging detector 212. Such method further includes causing the x-ray source or tube 210 to move along a source column to an initial height, wherein the initial height corresponds to the height of the x-ray imaging detector 212, and the source column supports the x-ray source 210 and is coupled to the floor surface at a first distance opposite the detector column. The method further includes causing the x-ray source 210 to emit an x-ray beam towards a center of the x-ray imaging detector 212 while the patient is positioned at least partially within a path of the x-ray beam; acquiring an x-ray image of the patient using the x-ray imaging detector 212; and during said acquiring, causing the x-ray source 210 to move between a plurality of positions along a curvilinear trajectory defined by an upper angular position, a home position, and a lower angular position.

In various embodiments, the computing device 228 may communicate with, or provide control signals to, the control unit 230 in order to complete one or more method steps, or communicate directly with the x-ray source 210 and the detector 212. Also in various embodiments, the computing device 228 may communicate with, or provide control signals to, positioning systems 238 and 240 in order to complete one or more method steps. For example, causing the x-ray source to move to the initial height may comprise sending a first control signal to the second positioning system 238 coupled to the x-ray source 210, the first control signal configured to cause vertical movement of the x-ray source 210 to the initial height. As another example, causing the x-ray source to move between the plurality of positions may comprise sending control signals to the positioning systems 238 and 240 to cause vertical and angular movement of the x-ray source.

In various embodiments, the x-ray source 210 is disposed at a first angle relative to a central axis of the x-ray imaging detector 212 when in the upper angular position and at a second angle relative to the central axis when in the lower angular position, the method further includes selecting the first angle and the second angle based on a height of the target area of the patient. In some embodiments, the method also includes adjusting the first distance between the detector column and the source column to a second distance based on a height of the target area of the patient prior to acquiring the x-ray image, and/or determining the detector height using a sensor configured to measure a vertical position of the x-ray imaging detector, wherein the first control signal is based on the measured position.

As referenced above, in various embodiments, the control unit 230 can control positioning and movement of various components of the upright imaging apparatus, including the detector 212 and the x-ray tube 210. In order to ensure precise, synchronous movement of all components, for example, during DTS image acquisition, each component may be electronically controlled by a set of three position control devices: a motor, a sensor, and a driver. The motor is an electronic device for mechanically or physically adjusting the position (e.g., vertical height and/or angle) of the component based on a signal received from the driver. The motor may be a servomotor or a brushless motor, for example. The sensor is an electronic device for measuring or detecting the actual position of the component (height and/or angle) and providing the actual position to the driver as an input signal. The sensor may be an encoder configured to provide absolute position information, for example. The driver is an electronic device that receives information (e.g., control signals) from the control unit 230 containing a required or desired positioning of the component and operates (or drives) the motor based thereon, while simultaneously reading inputs from the corresponding sensor, until the desired position is achieved. In some cases, each set of position control devices (collectively referred to herein as a “position control system”) is configured to control movement of the component along or relative to a single axis. Thus, for example, a component configured for axial movement in two directions may be controlled by two sets of devices.

Referring back to FIG. 5, a first position control system 236 (also referred to herein as a “detector position control system”) can be coupled to the x-ray detection device 208 for simultaneously controlling movement of the detector 212, as well as other components of the x-ray detection device 208 that are aligned with the detector 212, such as, e.g., the AEC chamber 224 and the anti-scatter grid 222. In order to synchronize movement of all three components, the first position control system 236 may be configured to move a housing of the x-ray detection device 208 (e.g., detector housing 109 shown in FIG. 1), rather than the individual components disposed therein. The first position control system 236 may be included in said housing of the device 208 or in an external support unit configured to movably connect the x-ray detection device 208 to the detector column. In some embodiments, the first position control system 236 may be included in, or implemented by, detector positioning system 107 shown in FIG. 1.

In embodiments, the first position control system 236 can be configured to control movement of the x-ray detection device 208 in a first axial direction defined by moving the device 208 vertically (i.e. up and down) or along a vertical axis of the device 208. Specifically, the first position control system 236 comprises a first motor 236a configured to control a vertical position, or height, of the x-ray detection device 208. The vertical position may be determined relative to a fixed constant, such as, for example, a bottom end of the detector column, the floor (e.g., floor surface 101) below the detector column, or other suitable location. The first position control system 236 also comprises a first driver 236b configured to receive control inputs from the control unit 230 that indicate a desired height or vertical position for the detector 212, or the x-ray detection device 208 as a whole. In some embodiments, the desired height may be automatically determined by the computing device 228 based on a height of the patient and/or the area of the patient to imaged, prior to beginning image acquisition, for example. In other embodiments, the desired height of the detector 212 may be manually entered (e.g., as discrete numeric values, as a selection from of a plurality of preset options, using buttons that move the detector up or down, etc.) by an operator of the apparatus 200 using the user interface 232. In still other embodiments, the x-ray detection device 208 may be manually or physically moved to a desired height by the operator or user of the apparatus 200.

The first position control system 236 further comprises a first sensor 236c configured to measure an actual height or vertical position of the x-ray detection device 208 and provide the measured value to the first driver 236b. The first driver 236b can be configured to compare the measured height to the desired height and determine whether further adjustment of the height is necessary to achieve the desired height. When the desired height is reached, the first driver 236b directs the first motor 236a to stop moving. In this manner, the detector 212, the AEC chamber 224, and the anti-scatter grid 222 can be jointly moved to a desired height. Though not shown, in some embodiments, the measurements taken by the first sensor 236c can be provided to the control unit 230 to guide or synchronize movement of other position control systems, such as, e.g., those coupled to the x-ray emission device 204.

As also shown in FIG. 5, a second position control system 238 (also referred to herein as “vertical source position control system”) can be coupled to the x-ray emission device 204 for controlling movement of the device 204 in the first axial direction (i.e. vertically), similar to the first position control system 236. More specifically, the second position control system 238 can be coupled to control movement of the x-ray emission device 204 along a vertical axis of the x-ray emission device 204 that is parallel to the vertical axis of the x-ray detection device 208. In addition, a third position control system 240 can also be coupled to the same device 204, but for controlling movement of the x-ray emission device 204 in a second axial direction defined by moving the device 204 relative to a horizontal axis of the x-ray emission device (such as, e.g., central axis 114 shown in FIG. 2), or tilting or rotating the device 204 about the horizontal axis.

In order to move the beam filtration mechanism 218, the collimator 217, and the DAP meter 220 in synchrony with the x-ray source 210, both the second position control system 238 and the third position control system 240 may be configured to move a housing of the x-ray emission device 204 (e.g., source housing 105 shown in FIG. 5), rather than the individual components disposed therein. The second and third position control systems 238 and 240 may be coupled to said housing of the device 204 or to an external support unit configured to rotatably connect the x-ray emission device 204 to the source column. In some embodiments, the two position control systems 238 and 240 may be included in, or implemented by, the source positioning system 111 shown in FIG. 1.

More specifically, the second position control system 238 comprises a second motor 238a configured to control a vertical position, or height, of the x-ray emission device 204, similar to first motor 236a. The second position control system 238 also comprises a second driver 238b configured to receive control inputs from the control unit 230 that indicate a desired height or vertical position for the x-ray source 210, or the x-ray emission device 204 as a whole, similar to the first driver 236b. The second position control system 238 further comprises a second sensor 238c configured to measure an actual height or vertical position of the x-ray emission device 204 and provide the measured value to the second driver 238b, similar to the first sensor 236c. The second driver 238b can be configured to compare the measured height to a presently desired height and if needed, instruct the motor 238a to keep moving. Once the driver 238b determines that the desired height has been reached, the second driver 238b can direct the second motor 238a to stop moving. In this manner, all components of the x-ray emission device 204 can be automatically moved to a desired height at the same time, or in one motion.

In some embodiments, the second position control system 238 is configured to track a vertical position of the detector 212, or the first position control system 236, and automatically adjust a position of the x-ray emission device 204 accordingly. In such cases, the “desired height” or goal provided to the second driver 238b may be a present height of the detector 212. In some embodiments, the present height of the detector 212 may be a height or vertical position value measured by the first position control system 236, or more specifically, the first sensor 236c, and provided to the control unit 230, for example, after the operator of the apparatus 200 has physically moved the x-ray detection device 208 to a height that vertically aligns the detector 212 with the object to be imaged. In other embodiments, the present height of the detector 212 may be a height or vertical position value received at the control unit 230 from the computing device 238, for example, after the operator of the apparatus 200 has entered or selected a desired detector height using the user interface 232. In either case, the control unit 230 provides a desired height value to the second driver 238b of the second position control system 238, and the second driver 238b causes the second motor 238a to move vertically until the vertical position detected by the second sensor 238c matches the desired height value. In this manner, a vertical height of the x-ray tube 210 can be automatically aligned with a present height of the detector 212 in real time.

The third position control system 240 (also referred to herein as an “angular source position control system”) comprises a third motor 240a configured to control an angular position of the x-ray emission device 204 by tilting or rotating the device 204 about the horizontal axis of the device 204 (e.g., towards or away from the detector column). The third position control system 240 also comprises a third driver 240b configured to receive control inputs from the control unit 230 that indicate a desired angle for the x-ray source 210, or the x-ray emission device 204 as a whole. The third position control system 240 further comprises a third sensor 240c configured to measure an actual angle or angular position of the x-ray emission device 204 and provide the measured value to the third driver 240b. The third driver 240b can be configured to compare the measured angle to a presently desired angle and if needed, instruct the motor 240a to keep tilting, until the driver 240b determines that the desired angle has been reached. Then the third driver 240b can instruct the third motor 240a to stop moving. In this manner, all components of the x-ray emission device 204 can be moved or tilted to a desired angle at the same time, or in one motion.

In embodiments, the second and third position control systems 238 and 240 may operate in two modes. In an initial mode, or prior to image acquisition, the desired height received from the control unit 230 at the second driver 238b may have been selected based on the height determined for the detector 212 according to a height of the patient. This desired height may determine a “home position” for that acquisition period, for example, like the home position shown in FIG. 2. Relatedly, the control unit 230 may send a zero value, or no angular position information, to the third driver 240b because the x-ray source 210 is positioned at a zero angle, or is not tilted, when in the home position, as shown in FIG. 2.

In a subsequent mode, or during image acquisition, the desired vertical position received from the control unit 230 at the second driver 238b may change several times, in very precise increments (e.g., millimeters), as the x-ray emission device 204 moves along a path prescribed for DTS acquisition. For example, as the device 204 follows the path shown in FIG. 2, the control unit 230 will send at least three control inputs to the driver 238b with respective vertical positions corresponding to the upper, home, and lower positions, as well as a plurality of intermediate height values corresponding to the spaces located between said positions along the path. Likewise, the control unit 230 will send, to the third driver 240b, a plurality of angular values, varying by a fraction of a degree in some cases, as the x-ray emission device 204 travels along the prescribed path during image acquisition. For example, as the device 204 follows the path shown in FIG. 2, the control unit 230 will send at least three control inputs to the driver 240b with respective angle values corresponding to the upper, home, and lower positions, as well as a plurality of intermediate angle values corresponding to the spaces located between said positions along the path. Moreover, the control unit 230 may be configured to synchronize a transmission of the height values to the second driver 238b with a transmission of the angle values to the third driver 240b, so that the x-ray emission device 204 can transition smoothly and quickly from one position to the next.

In some embodiments, the system 200 further comprises similar positioning devices (not shown) for moving one or more of the source column and the detector column closer together in order to accommodate patients of varying heights, as described herein. For example, in such cases, the system 200 may include a third position control system having a driver that is communicatively coupled to the control unit 230 for receiving a desired column separation value and/or other control inputs, a motor for controlling movement of the one or more columns in response to instructions from the driver, and a sensor for measuring actual position and providing the measured value to the driver.

According to embodiments, each of the x-ray detector 212 and x-ray tube 210 of the advanced imaging system 200 is capable of operating at very high speeds, which enables the overall system 200 to generate a large number of x-ray images during a given imaging cycle. More specifically, an overall operating speed of the advanced imaging system 200 may be determined, at least in part, by an image acquisition speed (or operational speed) of the detector 212. This operational speed (also referred to herein as “frame rate”) determines the threshold number of images that the detector 212 can generate per second. In embodiments, the x-ray detector 212 can be configured to acquire more than one image per second, such as, for example, about three to six images per second. In one exemplary embodiment, the detector 212 is configured to operate at a frame rate of about six images per second, such that the advanced imaging system 200 produces approximately 60 images during the time period in which the x-ray tube 210 moves from the top position to the bottom position (e.g., about 10 seconds). This time period, itself, may be selected based on how long a patient can hold his/her breath, as the patient must be extremely still during image acquisition.

An upper limit of the system's overall operating speed may be further determined by the speed at which the x-ray tube 210 can change positions while travelling along the path prescribed for DTS image acquisition, such as, for example, moving from the upper angular position to the next angular position along the path shown in FIG. 2. This position changing speed may be defined or determined by an operational speed of each positioning device used to move the x-ray emission device 204 from position to position along the prescribed path, i.e. the second and third position control systems 238 and 240 shown in FIG. 5. For example, the second position control system 238 may be configured to move vertically at an operational speed of about 10 cm per second, while the third positional control system 240 may be configured to move angularly at an operational speed of about three to four degrees per second. In some embodiments, the two position control systems 238 and 240 may be configured to cause simultaneous, or near simultaneous, vertical and angular displacement of the x-ray tube 210, to reduce the total amount of time required to move the x-ray tube 210 along the prescribed path. In such cases, the operational speeds of the second and third position control systems 238 and 240 may be synchronized, so that, for example, during each second, the x-ray tube 210 is moved both vertically and angularly.

According to embodiments, the operational speeds of the two position control systems 238 and 240 may also be synchronized with the operational speed, or frame rate, of the x-ray detector 212, in order to acquire a maximum number of images during the time that the x-ray tube 210 travels along the prescribed path. For example, in one embodiment, the prescribed path requires the x-ray tube 210 to travel vertically about one meter and angularly about 30 degrees. In such case, synchronizing the operational speeds of the position control systems 238 and 240 enables the x-ray tube 210 to travel the prescribed path in about 10 seconds, and synchronizing the operational speed of the x-ray detector 212 with that of the position control systems 238 and 240 enables the detector 212 to acquire about 60 images during the 10 second time period. Thus, the speed at which images are acquired by the detector-tube pair of system 200 may also be determined by the speed at which the x-ray source can travel or move.

In embodiments, this fact can be used to further improve the diagnostic benefits of the upright imaging system 200 by “oversampling” the projection images with different x-ray source settings during the DTS image acquisition process. In particular, during the time it takes for the x-ray tube 210 to change positions, an anode voltage setting of the x-ray beam may be switched from one energy value to another (e.g., high to low), and then back again during the next position change, so as to interlace, for example, high and low energy exposures with the different positions of the x-ray source. Such technique can create two or more independent data sets, depending on the number of different anode voltage settings, which can then be processed, by the computing device 228, for example, to obtain a differential image for each particular tomosynthesis slice image. For example, in the case of switching between two different anode voltage settings (e.g., high and low), two independent data sets may be created, and the differential image may be an image that represents a difference between a first image from the high anode voltage setting and a second image from the low anode voltage setting.

In addition, to enhance the spectral difference between x-ray pulses of different energies, a variable filtration mechanism can be applied to the x-ray beam so that the type of filtration changes from one exposure to another. More specifically, in embodiments, this can be achieved by inserting, in a pathway of the primary x-ray beam, a filter device comprising multiple filter materials and rotating the filter device in time with the x-ray pulse changes, so that a change in filtration material is synchronous with the change in x-ray pulse factor. In this manner, the advanced imaging system 200 can combine pulse-to-pulse switching of anode voltage (measured in kV) with simultaneous switching of the filter material to increase the difference in pulse-to-pulse spectra. In other embodiments, the two principles of pulse-to-pulse spectra variation (i.e. kV switching and filtering material changing) can be applied separately. In either case, by combining DTS and multi-energy imaging techniques, the system 200 is able to acquire information about the chemical composition of the structures being imaged for each DTS slice.

The beam filtration mechanism 218 shown in FIG. 5 is one example of the above-described filter device. Referring additionally to FIG. 6, shown is an exemplary embodiment of the beam filtration mechanism 218. In particular, the beam filtration mechanism 218 is depicted as a rotating disk with a plurality of filtration areas, each with a different filter material configured to provide a different spectral effect. As an example, the beam filtration mechanism 218 may include a first filtration area 242 comprising a first filter material selected from a group comprised of aluminum, copper, gold, silver, titanium, and tungsten, and a second filtration area 244 comprising a second, different filter material selected from the remainder of said group. As will be appreciated, a thickness of each filtration area can vary depending on the type of filter material used for that area. For example, an area comprised of aluminum may have a thickness of about 1.5 millimeters (mm) to about 4 mm, while an area comprised of silver may have a thickness of 100 micrometers (μm), and an area comprised of tungsten may have a thickness of a several micrometers.

In other embodiments, the beam filtration mechanism 218 may include more than two types of materials (and therefore, more than two filtration areas) and/or may have a different shape or configuration for each filtration area. Moreover, while the depicted embodiment shows the beam filtration mechanism 218 as a circular disc, in other embodiments, the beam filtration mechanism 218 may have a different overall shape, such as, e.g., a square, oval, rectangle, octagon, pentagon, hexagon, or any other suitable shape.

As shown in FIG. 5, operation of the beam filtration mechanism 218 may be controlled by the control unit 230. For example, the control unit 230 may send control signals to the beam filtration mechanism 218 for controlling a rotational speed of the mechanism during image acquisition. The speed of rotation may be adjusted or controlled so that the type of filter material placed in front of the x-ray beam, or within the beam path, changes from pulse to pulse, or for every two pulses. For example, in a first embodiment, the first filtration area 242 may cover or intersect the beam path during a first pulse containing high anode voltage (or energy), and the second filtration area 244 may cover or intersect the beam path during a second pulse containing low anode voltage. To achieve this level of synchronization, the control unit 230 may be configured to set the rotational speed of the beam filtration mechanism 218 according to the rate at which the anode voltage settings are changed from pulse to pulse, which, as described above, is determined based on the source positioning speed (i.e. the speed at which the x-ray emission device 204 changes position). Alternatively, in a second embodiment, the speed of rotation may be configured so that the first filtration area 242 remains in the beam path during a first set of high and low energy pulses, and the second filtration area 244 intersects the beam path during a second set of high and low energy pulses.

Referring back to FIG. 5, in embodiments, the advanced imaging system 200 is capable of operating in several different image acquisition modes, such as, for example, a DTS imaging mode, a dynamic imaging mode, a multi-energy or other spectral imaging mode, a classical x-ray mode, or a combination mode that combines two or more of these imaging modes. In some embodiments, the user interface 232 can be configured to enable user selection of an available image acquisition mode, and the computing device 228 can be configured to control operation of the system 200 in accordance with the selected mode, for example, by launching a software application configured to control the upright imaging apparatus in accordance with the selected imaging mode.

FIGS. 7 through 9 are flowcharts of exemplary data acquisition and processing steps (or methods) that may be performed by the advanced imaging system 200 while operating in a selected one of three exemplary imaging modes: the DTS mode, the multi-energy mode, and a third mode that combines both, in accordance with embodiments. Each of the methods (i.e. methods 300, 400, and 500) can be implemented, at least in part, by at least one data processor executing software stored in a memory, such as, for example, the processor and memory included in the computing device 228 of system 200 shown in FIG. 5. In order to carry out the operations of a given method 300/400/500, the computing device 228 may interact with one or more other components of the system 200, such as, for example, the HV generator 226, the user interface 232, the detector controller 234, and the control unit 230, and the control unit 230, in turn, may interact with the first, second, and/or third position control systems 236, 238, and 240 and/or the beam filtration mechanism 218.

Referring now to FIG. 7, shown is an exemplary method 300 of carrying out a DTS mode of operation to obtain diagnostic images, in accordance with embodiments. The method 300 begins at step 302 with movement of the x-ray tube 210 along a DTS trajectory, such as, e.g., the prescribed path shown in FIG. 2. Such vertical and/or angular movement of the x-ray tube 210 may be achieved by the control unit 230 sending appropriate control signals to the x-ray emission device 204, or more specifically, the second and third position control systems 238 and 240 coupled thereto. Step 302 further includes, emission of x-ray pulses from the x-ray tube 210 as the x-ray emission device 204 moves along the trajectory, and registration of said pulses by the detector 212. The frequency of image acquisition at the detector 212 may be determined by the speed of positioning of the x-ray tube 210 and the available image acquisition frequency of the detector 212, as described herein.

Due to the process in step 302, at step 304, a set of raw multi-position projections is created and, in some cases, stored in a memory. The memory may be, for example, one associated with, or included in, the computing device 228 of the advanced imaging system 200. At step 306, pre-processing techniques are applied to the projectional images obtained at step 304 by one or more processors (e.g., an image processor and/or data processor) included in the computing device 228, in order to improve a quality of the images.

The pre-processing techniques applied at step 306 can include, for example, detector corrections to remove or correct for flood, dark, and dead pixels. For example, gain correction may be necessary to correct flooding, or to account for each pixel in the detector 212 having its own sensitivity. Removal of dead pixels may be achieved using an algorithm that analyzes each image acquired from the detector 212, identifies pixel values in those images that do not correspond to the intensity of the x-ray beam, and replaces the identified pixel value with an appropriate value. Dark noise removal may involve identifying and removing electrical signals generated by the detector 212 when it is not irradiated by the x-ray beam.

The pre-processing step 306 may also, or alternatively, include, scatter correction. Compton scatter results in a degradation of the image quality, which results in a loss of contrast resolution and non-quantitative values. One technique for compensating for this effect is to add the anti-scatter grid 222 in the path of the x-ray beam (e.g., as shown in FIG. 5), but this can increase the radiation dose delivered to the patient. At step 306, scatter correction may be achieved for example, based on processing or deep learning techniques, instead of using the grid 222.

In multi-image protocols (e.g., multiple energy, digital subtraction angiography, tomosynthesis, etc.), respiratory and cardiac motion and other patient movements result in artifacts and a loss of spatial resolution. Accordingly, the pre-processing step 306 may also, or alternatively, include, motion correction, which may be achieved using one or more existing techniques.

At step 308, tomosynthesis reconstruction may be completed using the processed images obtained at step 306. Due to the finite size of the detector 212, tomosynthesis techniques can result in artifacts derived from the truncation of the projection. Thus, step 308 can also include applying truncation correction pre-processing techniques, by the one or more processors of the computing device 228, to compensate for this limitation. One of several alternative algorithms may be used to reconstruct the tomosynthesis images at step 308, such as, for example, deep learning method, “shift & add” method, high boost filtering, and iterative reconstruction, as will be appreciated. In the latter case, prior information may be used to compensate for the lack of projection data when obtaining the tomosynthesis image. At step 310, the final tomosynthesis image is obtained by the one or more processors and may be stored, displayed, and/or output for diagnostic purposes.

Referring now to FIG. 8, shown is an exemplary method 400 of carrying out a multi-energy mode of operation to obtain diagnostic images, in accordance with embodiments. The method 400 begins at step 402 with x-ray pulse generation, along with spectrum variation and acquisition, as shown. More specifically, the HV generator 226 generates high voltage pulses and provides those pulses to the x-ray tube 210. In addition, the HV generator 226 changes the anode voltage level or energy level from one pulse to the next, so as to create an alternating pattern of high and low energy levels. At the same time, the system 200 activates the beam filtration mechanism 218 in order to place various filter materials in the path of the x-ray beam and thereby, additionally vary the spectra of the x-ray beam. As a result, a sequence of x-ray pulses with different spectra can be generated at step 402. This sequence passes through the patient and is captured by the detector 212.

Still referring to step 402, the exact voltage levels used for a given anode voltage pair may be pre-selected based on the region or organ of the patient body to be imaged. For example, typical values for chest imaging include a high anode voltage level of 120 kilovolts (kV) and a low anode voltage level of 60 kV. Generally speaking, the low energy level will be as low as possible but still high enough to penetrate the area of interest on the patient's body (e.g., below 80 kV), and the high energy level will be the standard kV value that is used for non-spectral imaging, as will be appreciated.

At step 404, a resulting series of projectional raw multi-energy images is acquired and stored in the memory of the computing device 228. At step 406, images are processed by one or more processors of the computing device 228 using a pre-processing algorithm to improve the quality of each individual image. The pre-processing step 406 may be similar to the pre-processing step 306 shown in FIG. 7 and described herein.

At step 408, material decomposition techniques are applied by the one or more processors to acquire quantitative information about the chemical composition of the patient's anatomy, or the area through which the x-ray beam has passed. One technique includes applying a material decomposition separation algorithm to acquire quantitative information about the chemical materials in said area. To perform this separation, prior acquired spectral calibration information may be compared to the presently acquired information. Another technique includes applying a deep-learning material decomposition algorithm to improve the separation between different materials. Through these techniques, a quantitative planar image of the patient's anatomy can be acquired at step 410. The acquired image(s) can be stored in the memory of the computing device 228.

FIG. 9 illustrates an exemplary method 500 of carrying out a joint DTS and multi-energy mode of operation to obtain diagnostic images, in accordance with embodiments. The method 500 begins at step 502 with varying x-ray spectra by changing the parameters of the high voltage pulse provided to the x-ray tube 210 and applying various filtration materials to the x-ray beam using the beam filtration mechanism 218, for example, similar to step 402 of method 400. The method 500 also includes, at step 503, movement of the x-ray tube 210 along a DTS trajectory while simultaneously emitting x-ray pulses and registering said pulses at the detector 212, similar to step 302 of method 300. Steps 502 and 503 may be carried out simultaneously, in close succession, or in conjunction, according to various embodiments.

Based on the activities from steps 502 and 503, a resulting set of raw, multi-energy, multi-position projections are acquired and stored in a memory, at step 504. Then, at step 506, pre-processing techniques similar to pre-processing step 306 of method 300 are applied to the projections, by the one or more processors, to improve the quality of each individual image in the set.

Next, at step 508, a tomosynthesis reconstruction algorithm similar to step 308 of method 300 is applied to the images produced at step 506 by the one or more processors. In particular, the tomosynthesis reconstruction algorithm is applied individually to the image resulting from each combination of high voltage pulse energy level and filter material applied at step 502 to produce a tomosynthesis image or slice. In embodiments, the exact number or amount of images obtained can be equal to the number of energy level and filter material combinations used during the raw images acquisition process at step 504. The resulting set of tomosynthesis images obtained at step 508 can represent an optical density distribution of the object being imaged for the particular spectral characteristics of the x-ray beam, or how transparent the object is to different incident radiation. As will be appreciated, this density information depends on the properties of the object (e.g., its chemical composition and size), as well as the properties of the incident radiation (e.g., spectral value).

At step 510, a material separation algorithm similar to step 408 of method 400 is applied, by the one or more processors, to the same spatially-positioned slices acquired at different voltage pulses and/or filter combinations, i.e. all slices positioned at the same location but obtained using different voltage and filter settings. At step 512, quantitative tomosynthesis images are obtained that represent quantitative information about the chemical composition of the patient body in one particular slice. By combining DTS with multi-energy techniques, these slices of the patient anatomy provide not only optical density information (as in conventional DTS), but also chemical composition information. In this manner, the resulting images can provide a radiologist or other medical professional reading the images with more information about the internal structures of the patient, which increases the overall sensitivity of the method 500 to detecting a given disease than conventional DTS systems.

In other embodiments, an order in which the tomosynthesis reconstruction algorithm, or step 508, and the material separation algorithm, or step 510, are performed may be switched without affecting the images ultimately produced by the method 500. For example, in such cases, the material separation algorithm may be applied to the pre-processed images produced at step 506, so as to create a set of quantitative planar images representing the chemical composition of the scanned patient anatomy, for example, similar to step 410 of method 400. The planar images can then be processed using the tomosynthesis reconstruction algorithm at step 508 to achieve the set of slices at step 512, thus still representing a chemical composition of the patient anatomy in that particular slice.

Referring back to FIG. 5, according to embodiments, the computing device 228 can be a personal computer (e.g., desktop, laptop, tablet-type, or otherwise), a special or general purpose digital computer (such as a mainframe computer), a workstation, a minicomputer, a computer network, a “virtual network,” a “internet cloud computing facility,” a mobile or handheld computer (e.g., personal digital assistant, smartphone, tablet, etc.), or any another suitable device.

The memory of the computing device 228 can be any appropriate memory device suitable for storing software instructions, such as, for example, a volatile memory element (e.g., random access memory (RAM, such as DRAM, SRAM, SDRAM, etc.)), a nonvolatile memory element (e.g., ROM, hard drive, tape, CDROM, etc.), or any combination thereof. Moreover, the memory may incorporate electronic, magnetic, optical, and/or other types of storage media. In some embodiments, the memory includes a non-transitory computer readable medium for implementing all or a portion of one or more of the methods described herein and shown in FIGS. 7 through 9.

The memory can store one or more executable computer programs or software modules comprising a set of instructions to be performed, such as, for example, one or more software applications that may be executed by the at least one processor to carry out the principles disclosed herein (e.g., methods 300, 400, and/or 500). The executable programs can be implemented in software, firmware, hardware, or a combination thereof. In some cases, the memory is also utilized to implement at least part of one or more databases utilized by the advanced imaging system 200, such as, for example, an x-ray imaging database for storing x-ray images and/or information related thereto.

The at least one processor of the computing device 228 can be any appropriate hardware device for executing software instructions retrieved from the memory, such as, for example, a central processing unit (CPU), a semiconductor-based microprocessor (in the form of a microchip or chip set), or another type of microprocessor. In some cases, the at least one processor includes an image processor for collecting, processing, and enhancing an x-ray image signal or other information received from the detector controller 234, and the memory is configured to store the processed image.

Thus, an upright advanced imaging system for generating images of the internal structures of a human body is provided with a first vertical column (or source column) configured to hold an x-ray source and an electrical motor coupled to the x-ray source for rotating the x-ray source relative to the first vertical column and for adjusting a height of the x-ray source. The system further comprises a second vertical column (or detector column) configured to hold an x-ray detector (or receptor) that is capable of capturing more than one image per second. The system also comprises a high voltage generator capable of generating more than one level of high voltage pulse per second and supplying each pulse towards an x-ray tube. The x-ray source can be configured to rotate about, or relative to, a horizontal axis at an angle selected from about −20 degrees to about +20 degrees, where at about 0 degrees the x-ray beam is perpendicular to a surface of the x-ray detector. The advanced imaging system also comprises a position control system configured to rotate the x-ray source such that a central x-ray beam remains aimed at a center of the x-ray detector.

According to aspects of the upright advanced imaging system, the x-ray detector, the x-ray source, and the high voltage generator may be synchronized such that the x-rays are generated in the period when the x-ray detector captures an x-ray projection image. Moreover, multiple x-ray projection images may be captured with simultaneous vertical movement of the x-ray source.

Also, according to aspects of the upright advanced imaging system, for each pulse, the high voltage generator may be capable of varying one or more of a plurality of adjustable x-ray pulse parameters, the parameters comprising one or more of anode voltage, anode current, and length of the pulse. Moreover, for each pulse, a beam filtration mechanism may be activated or implemented in order to change the x-ray beam filtration from pulse to pulse.

The system further includes a computing device (e.g., personal computer) configured to control various aspects of the upright advanced imaging system. The computing device comprises a memory configured to store the x-ray projection images received at the detector, and an algorithm configured to convert the x-ray projection images into a set of images representing an anatomy of the patient in multiple planes, parallel to a surface of the x-ray detector (i.e. slices).

According to certain aspects, a distance between the first vertical column and the second vertical column is selected from a range of about 1 meter to about 2.2 meters depending on a height of the patient and/or the target area of the patient. According to other aspects, the range of permissible vertical movement of the x-ray source during image acquisition is determined based on a height of the patient and/or target area.

In certain embodiments, the process descriptions or blocks in the figures, such as FIGS. 7, 8, and 9, can represent modules, segments, or portions of code which include one or more executable instructions for implementing specific logical functions or steps in the process. Any alternate implementations are included within the scope of the embodiments described herein, in which functions may be executed out of order from that shown or discussed, including substantially concurrently or in reverse order, depending on the functionality involved, as would be understood by those having ordinary skill in the art.

It should be emphasized that the above-described embodiments, particularly, any “preferred” embodiments, are possible examples of implementations, merely set forth for a clear understanding of the principles of the invention. Many variations and modifications may be made to the above-described embodiment(s) without substantially departing from the spirit and principles of the techniques described herein. All such modifications are intended to be included herein within the scope of this disclosure and protected by the following claims.

1. An x-ray imaging apparatus, comprising:
an x-ray source for emitting an x-ray beam towards a center of an x-ray imaging detector;
the x-ray imaging detector configured to acquire an x-ray image of a patient positioned adjacent to the x-ray imaging detector and at least partially within a path of the x-ray beam;
a first vertical column attached to a floor surface and configured to support the x-ray source;
a second vertical column configured to support the x-ray imaging detector and attached to the floor surface at a first distance opposite the first vertical column, the x-ray image detector being adjustably positioned along an extent of the second vertical column at a detector height configured to substantially align with a target area of the patient;
a positioning system configured to control vertical and angular movement of the x-ray source relative to the first vertical column, wherein:
prior to image acquisition, the positioning system is configured to move the x-ray source to an initial height determined based on the detector height, and
during image acquisition, the positioning system is configured to move the x-ray source to a plurality of positions along a trajectory defined by an upper angular position, a home position, and a lower angular position.

2. The x-ray imaging apparatus of claim 1, wherein the trajectory is configured for image acquisition using digital tomosynthesis (DTS).

3. The x-ray imaging apparatus of claim 1, wherein in the home position, the x-ray source is disposed at the initial height, and the x-ray beam is directed perpendicular to a front surface of the x-ray imaging detector.

4. The x-ray imaging apparatus of claim 1, wherein:
in the upper angular position, the x-ray source is disposed above a central axis of the x-ray imaging detector, and the x-ray beam is directed towards a center of the detector from a first angle relative to the central axis; and
in the lower angular position, the x-ray source is disposed below the central axis of the x-ray imaging detector, and the x-ray beam is directed towards the detector at a second angle relative to the central axis of the detector.

5. The x-ray imaging apparatus of claim 4, wherein the first angle and the second angle are equal in magnitude.

6. The x-ray imaging apparatus of claim 5, wherein the magnitude of the first and second angles is selected based on a height of the target area of the patient.

7. The x-ray imaging apparatus of claim 1, wherein the first distance between the first vertical column and the second vertical column is adjustable to a second distance depending on the height of the target area of the patient.

8. The x-ray imaging apparatus of claim 1, further comprising a control unit configured to:
send a first control signal to the positioning system for moving the x-ray source to the initial height prior to image acquisition; and
send second and third control signals to the positioning system, in synchrony, to cause vertical and angular movement of the x-ray source relative to the x-ray imaging detector during image acquisition.

9. The x-ray imaging apparatus of claim 8, wherein the positioning system includes a first position control system comprising:
a first motor configured to adjust a vertical position of the x-ray source;
a first driver configured to receive corresponding control signals from the control unit and drive the first motor based thereon, each control signal indicating a desired vertical position for the x-ray source; and
a first sensor configured to measure an actual vertical position of the x-ray source and provide the actual position to the first driver, the first driver being configured to stop movement of the first motor once the actual position matches the desired position.

10. The x-ray imaging system of claim 9, wherein the positioning system further includes a second position control system comprising:
a second motor configured to adjust an angular position of the x-ray source;
a second driver configured to receive corresponding control signals from the control unit and drive the second motor based thereon, each control signal indicating a desired angular position for the x-ray source; and
a second sensor configured to measure an actual angular position of the x-ray source and provide the actual position to the second driver, the second driver being further configured to stop movement of the second motor once the actual position matches the desired position.

11. An x-ray imaging system, comprising:
an x-ray emission device comprising an x-ray source for emitting an x-ray beam towards a center of an x-ray imaging detector;
an x-ray detection device comprising the x-ray imaging detector for acquiring an x-ray image of a patient positioned adjacent to the x-ray imaging detector and at least partially within a path of the x-ray beam;
a positioning system configured to control vertical and angular movement of the x-ray emission device;
a control unit configured to send control signals to the positioning system during image acquisition to move the x-ray emission device along a curvilinear trajectory about the x-ray imaging detector; and
an x-ray generator configured to provide high voltage pulses of two or more different energy levels to the x-ray source for generating the x-ray beam, the x-ray generator being further configured to change from a first energy level to a second energy level while the positioning system moves the x-ray emission device from one position along the trajectory to a next position along the trajectory.

12. The x-ray imaging system of claim 11, wherein an image acquisition speed of the x-ray imaging detector is determined by a speed at which the positioning system changes the position of the x-ray emission device.

13. The x-ray imaging system of claim 11, further comprising a beam filtration mechanism having a plurality of filter materials, the beam filtration mechanism being configured to place a selected one of the filter materials within the path of the x-ray beam during each pulse.

14. The x-ray imaging system of claim 13, wherein the beam filtration mechanism is configured to rotate at a second speed to change the filter material placed in the path of the x-ray beam, the second speed being selected based on the speed at which the positioning system changes the position of the x-ray emission device.

15. The x-ray imaging system of claim 14, wherein the control unit is further configured to send control signals to the beam filtration mechanism for controlling said rotation.

16. The x-ray imaging system of claim 13, wherein the plurality of filter materials includes a first filter material and a second filter material, and the second speed is selected so that the first filter material intersects the path of the x-ray beam during emission of a pulse at the first energy level, and the second filter material intersects the path of the x-ray beam during emission of a pulse at the second energy level.

17. A method, comprising:
setting a detector height for an x-ray imaging detector supported by a detector column attached to a floor surface, the detector height configured to substantially align with a target area of a patient positioned adjacent the x-ray imaging detector;
causing an x-ray source to move along a source column to an initial height, the initial height corresponding to the height of the x-ray imaging detector, wherein the source column supports the x-ray source and is coupled to the floor surface at a first distance opposite the detector column;
causing the x-ray source to emit an x-ray beam towards a center of the x-ray imaging detector while the patient is positioned at least partially within a path of the x-ray beam;
acquiring an x-ray image of the patient using the x-ray imaging detector; and
during said acquiring, causing the x-ray source to move between a plurality of positions along a curvilinear trajectory defined by an upper angular position, a home position, and a lower angular position.

18. The method of claim 17, wherein the trajectory is configured for image acquisition using digital tomosynthesis (DTS).

19. The method of claim 17, wherein the x-ray source is disposed at a first angle relative to a central axis of the x-ray imaging detector when in the upper angular position, and is disposed at a second angle relative to the central axis when in the lower angular position, the method further comprising: selecting the first angle and the second angle based on a height of the target area of the patient.

20. The method of claim 17, further comprising: adjusting the first distance between the detector column and the source column to a second distance based on a height of the target area of the patient prior to acquiring the x-ray image.

21. The method of claim 17, wherein causing the x-ray source to move to the initial height comprises: sending a first control signal to a positioning system coupled to the x-ray source, the first control signal configured to cause vertical movement of the x-ray source to the initial height.

22. The method of claim 21, further comprising: determining the detector height using a sensor configured to measure a vertical position of the x-ray imaging detector, wherein the first control signal is based on the measured position.

23. The method of claim 21, wherein causing the x-ray source to move between the plurality of positions comprises sending control signals to the positioning system to cause vertical and angular movement of the x-ray source.
